(19) United States
(12) Patent Application Publication
(10) Pub. No.: US 2016/0106330 A1
(43) Pub. Date: Apr. 21, 2016

(54) CARDIAC MONITORING DEVICE AND METHOD

(71) Applicant: ECM Foresights, LLC, Warsaw, IN (US)

(72) Inventors: Matthew C. Abernethy, Warsaw, IN (US); Mohammad I. Dotani, Oklahoma City, OK (US); Amit Prakash, Buffalo Grove, IL (US)

(73) Assignee: ECM Foresights, LLC, Warsaw, IN (US)

(21) Appl. No.: 14/885,384

(22) Filed: Oct. 16, 2015

Related U.S. Application Data
(60) Provisional application No. 62/065,072, filed on Oct. 17, 2014.

(51) Int. Cl. A61B 5/04; A61B 5/00; A61B 5/0452; A61B 5/02

(52) U.S. Cl. CPC A61B 5/04012; A61B 5/02; A61B 5/6801; A61B 5/0006; A61B 5/746; A61B 5/4836; A61B 5/0452; A61B 2560/0475

(57) ABSTRACT

A method of predicting cardiac disease includes the steps of: attaching a monitoring device to a patient; obtaining cardiac information from the patient using the monitoring device; transmitting the obtained cardiac information from the monitoring device to a data-computing device; analyzing the transmitted cardiac information using the data-computing device and a predictive model; building an indication report from the analyzed cardiac information; and outputting the indication report.

### CROSS REFERENCE TO RELATED APPLICATIONS

This is a non-provisional application based upon U.S. provisional patent application Ser. No. 62/065,072, entitled “CARDIAC MONITORING DEVICE AND METHOD”, filed Oct. 17, 2014, which is incorporated herein by reference.

### BACKGROUND OF THE INVENTION

1. Field of the Invention

The present invention relates to medical monitoring devices, and, more particularly, to cardiac monitoring devices and methods.

2. Description of the Related Art

Cardiovascular disease is a class of diseases that involve the heart or blood vessels. It is common knowledge that many underlying causes of cardiovascular disease are tied to lifestyle choices. For example, in nations where sedentary lifestyles have become increasingly common, the observed incidence of cardiovascular disease has increased. Known risk factors for cardiovascular disease have been found to be age, gender, tobacco use, physical fitness, and diet.

Cardiovascular disease can be classified as either chronic or acute. Chronic cardiovascular disease is a relatively slow and progressive loss of cardiac function, whereas acute cardiovascular disease is a sudden loss of cardiac function. While chronic cardiovascular disease can be monitored at regular intervals to determine if there has been any progression in cardiac function loss and determine an appropriate treatment plan, acute cardiovascular disease tends to be caused by triggering events that are unknown or cannot be reliably predicted. As such, treatment of acute cardiovascular disease usually involves general preventive measures by addressing a patient's risk factors associated with cardiovascular disease, such as obesity, and immediate treatment following an acute cardiovascular disease event. Further, many diagnoses of cardiac events or pending cardiac events are inaccurate, which leads to unnecessary surgery and/or the use of invasive diagnostic procedures to confirm or, more commonly, discredit an initial diagnosis. The large inaccuracy in diagnosing pending or occurring cardiac events therefore leads to a significant amount of unnecessary invasive diagnostic and surgical procedures that increases a patient's risk of experiencing medical complications with no medical benefit.

What is needed in the art is a more reliable way to predict and prevent cardiovascular disease.

### SUMMARY OF THE INVENTION

The present invention provides a device and method for determining a patient's risk of cardiac disease by collecting cardiac information from the patient and using the collected information to build an indication report for the patient's risk of cardiovascular disease.

The invention in one form is directed to a method of predicting cardiac disease including the steps of: attaching a monitoring device to a patient; obtaining cardiac information from the patient using the monitoring device; transmitting the obtained cardiac information from the monitoring device to a data-computing device; analyzing the transmitted cardiac information using the data-computing device and a predictive model; building an indication report from the analyzed cardiac information; and outputting the indication report.

The invention in another form is directed to a method of maintaining a cardiac disease data repository including the steps of: storing baseline data in a server including at least one memory unit, the baseline data including a plurality of patient cardiac states and a cardiac event indicator associated with at least one of the patient cardiac states; receiving an incoming patient cardiac state from a monitoring device attached to a patient, the server receiving the incoming patient cardiac state; receiving an incoming cardiac event indicator associated with the incoming patient cardiac state, the server receiving said incoming cardiac event indicator; and updating the baseline data to store the incoming patient cardiac state and the associated cardiac event indicator in the at least one memory unit.

An advantage of the present invention is a patient's near-term risk of a cardiac event occurring can be predicted from previously obtained and analyzed cardiac information.

Another advantage is a data repository can be built from cardiac information collected from multiple patients.

Yet another advantage is the data repository can be utilized for statistical analysis to produce increasingly effective predictive models.

Yet another advantage is more accurate diagnoses of cardiac events can be obtained and decrease the number of unnecessary surgical and invasive diagnostic procedures performed on patients.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a flow chart of an embodiment of a method according to the present invention;

FIG. 2 is a front view of a patient with the heart illustrated and various electrodes placed on the patient's body;

FIG. 3 is a wave-form diagram of cardiac information that can be obtained from a patient;

FIG. 4 is a schematical representation of a monitoring device used to gather signals and/or data for manipulation by embodiments of the present invention;

FIG. 5 is an illustration of a patient wearing a monitoring device which is in communication with a remote data center;

FIG. 6 is a schematical representation of a treatment device incorporating the monitoring device shown in FIG. 4;

FIG. 7 is a flow chart of another embodiment of a method according to the present invention; and

FIG. 8 is an illustration of an example indication report that can be output according to the method illustrated in FIG. 1.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

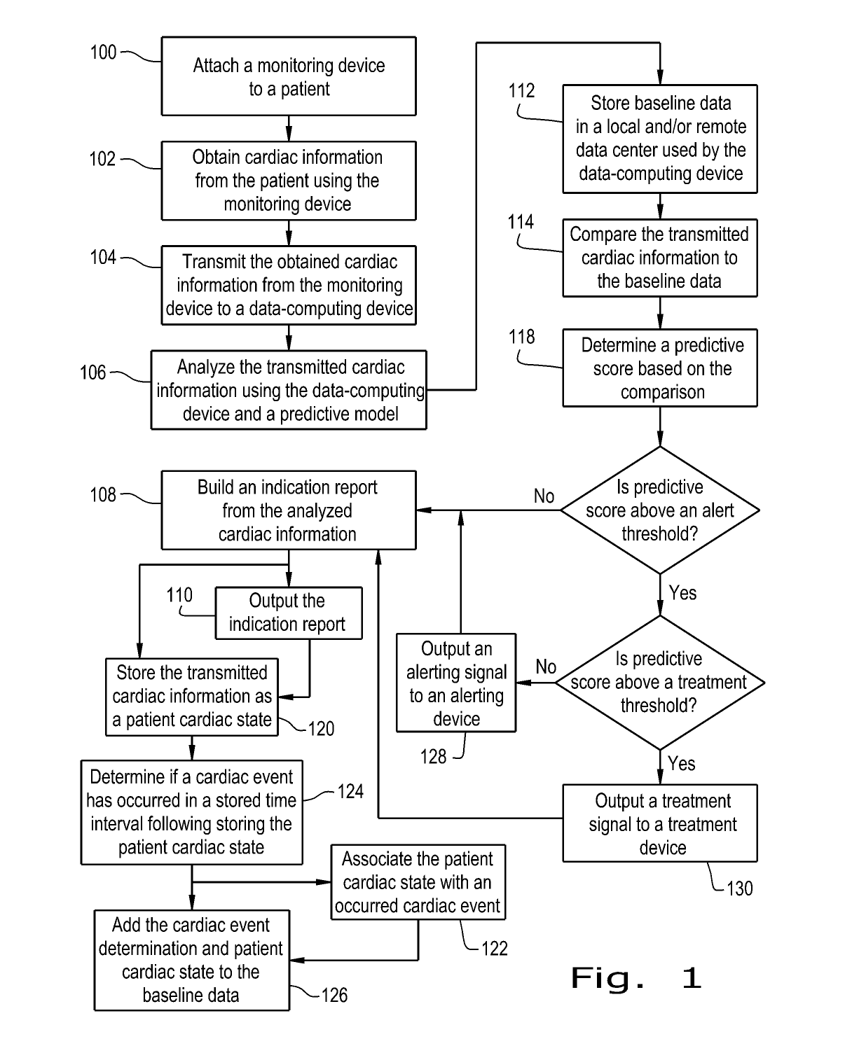

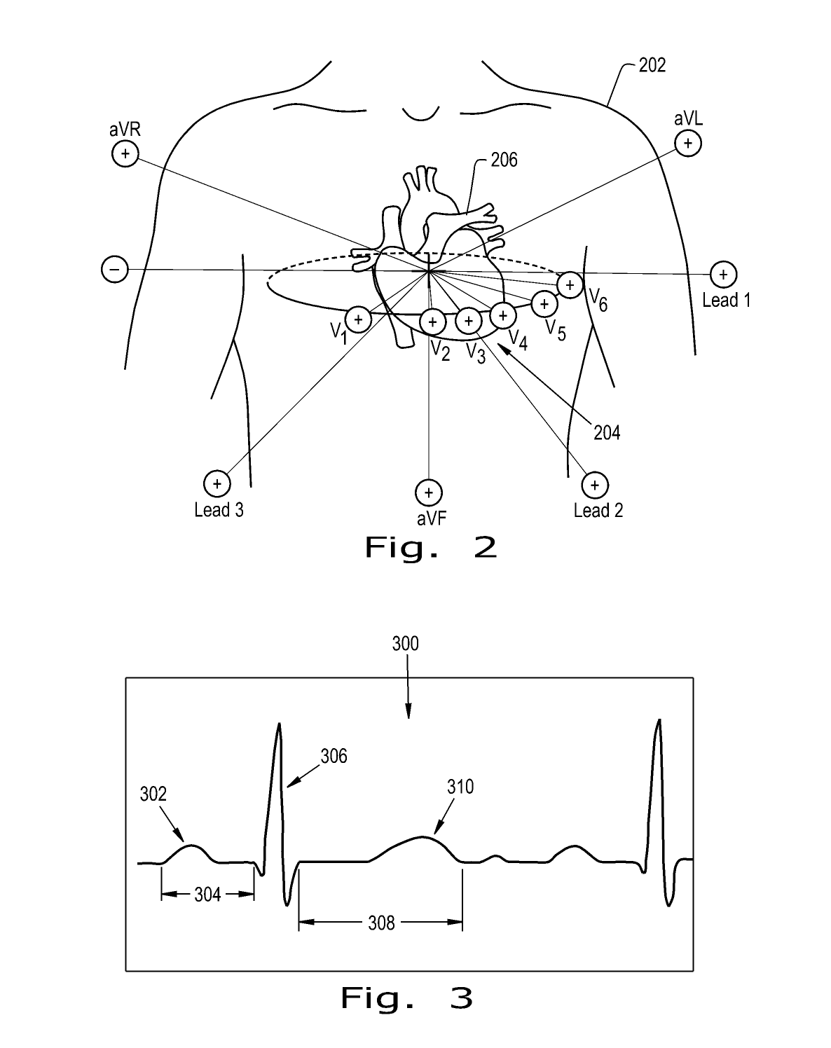

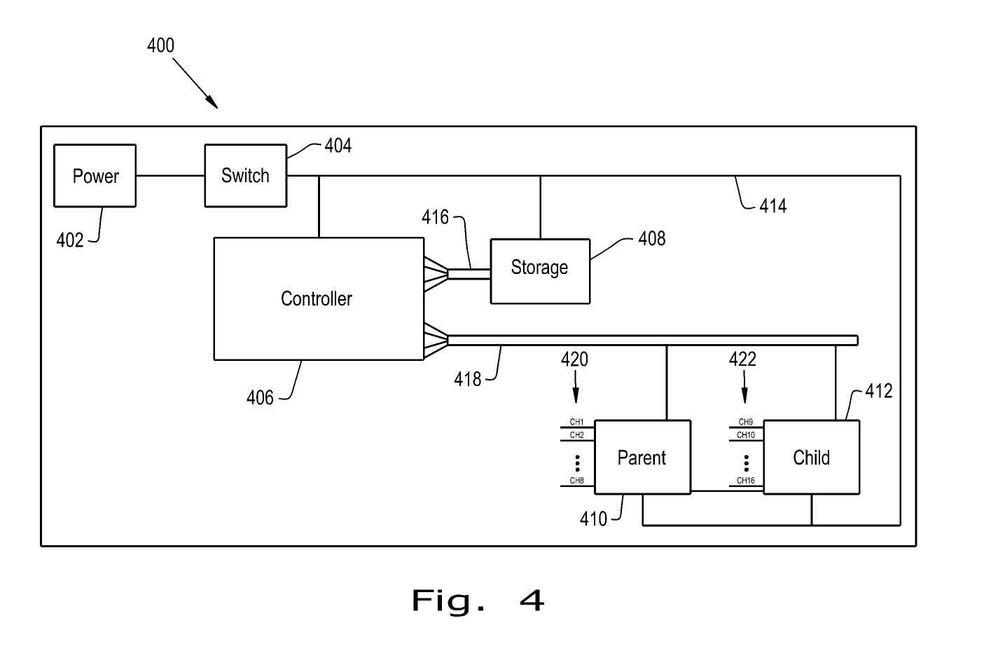

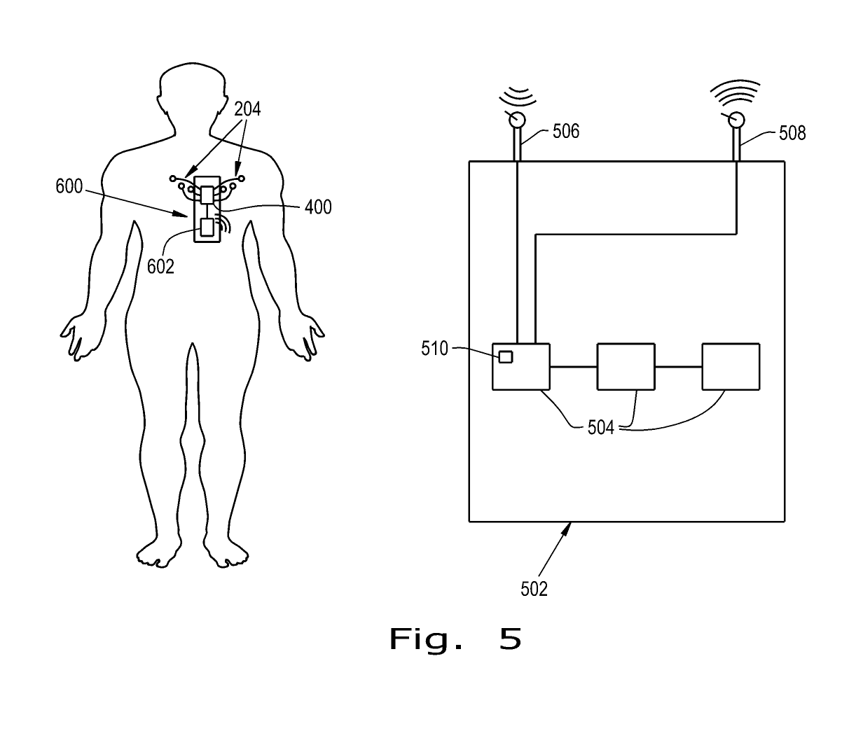

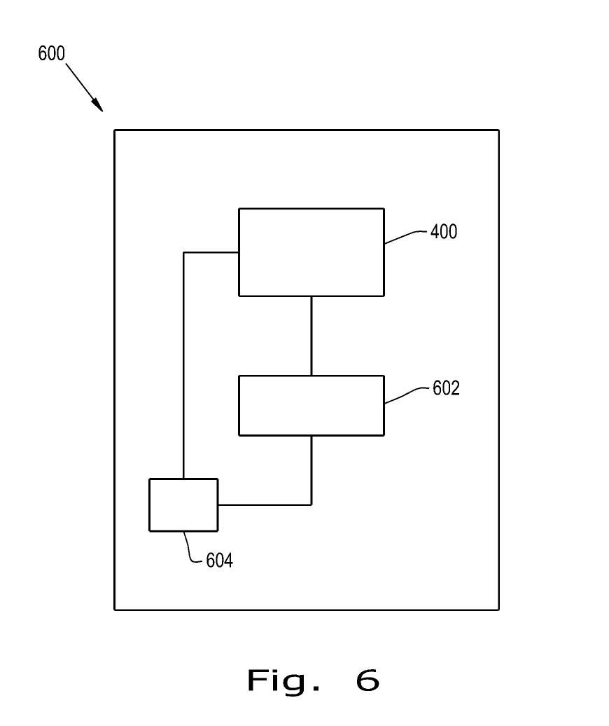

Referring now to the drawings, and more particularly to FIGS. 1-6, there is shown a flow chart which generally demonstrates the steps of attaching 100 a monitoring device 400 (shown in FIG. 4) to a patient 202 (shown in FIG. 2), obtaining 102 cardiac information from the patient 202 using the monitoring device 400, transmitting 104 the obtained cardiac information from the monitoring device 400 to a data-computing device 504 (shown in FIG. 5), analyzing 106 the transmitted cardiac information using the data-computing device 504, building 108 an indication report from the analyzed cardiac information, and outputting 110 the indication report built from the analysis. Optionally, the information can be stored and analyzed to contribute to an aggregate analysis or baseline data. The monitoring device 400 can be any type of device commonly used to detect cardiac activity. Such devices can include or be connected to electrodes 204 (shown in FIG. 2), wires, patches, stickers, nodes, imaging devices, etc. It is useful if the monitoring device 400 is non-invasive, i.e., an incision is not necessary to implant the monitoring device 400 in a patient, but it is contemplated that an invasive monitoring device can also be used, such as an implanted pacemaker. The monitoring device 400 does not need to be a device that is intended solely to collect and transmit cardiac information, but can be a multi-purpose device such as a computer, tablet, smart phone, or other device that includes an electrical processing circuit. Similarly, the monitoring device 400 can be incorporated in a treatment device 600 (shown in FIG. 6) which has multiple components that are connected and/or coupled to each other, such as a plurality of electrodes that are incorporated in a shirt worn by a patient that connect to the monitoring device 400. It should be understood that the monitoring device 400 shown in FIG. 4 and described herein is only one example of such a monitoring device that can be used to detect cardiac electrical signals and that the principles of the present invention can be readily applied to other monitoring devices.

Referring now to FIG. 2, electrodes 204 that can couple to the monitoring device 400 are shown attached to a patient 202. It should be appreciated that sensors other than electrodes can be coupled to the monitoring device 400 and attached to the patient 202 to collect cardiac information. As can be seen, the electrodes 204 are attached to the patient 202 at various points along the torso. As shown, the electrodes 204 are placed all along the torso so that data can be collected along a path through the electrodes 204 that extends 360 degrees around the patient's heart 206. The electrodes 204 can be aligned along the torso in any configuration that allows for a sufficiently clear cardiac data signal to be produced that does not have an amount of artifacts that prevent the cardiac data signal from being interpreted. As shown in FIG. 2, the electrodes 204 are arranged along a circumference of a circle that is roughly defined by a radius that extends from the patient's heart 206. The various electrodes 204 used can include an automated volt right 204A, an automated volt left 204B, an automated volt foot 204C, a lead I 204D, a lead II 204E, and a lead III 204F, as shown. Such an arrangement can capture cardiac electrical signals as they propagate across the heart 206. Other arrangements of electrodes 204 are also contemplated and can include electrodes placed on the extremities of a patient and other regions of the torso, among other regions. It is similarly contemplated that fewer electrodes can be used, such as 3 electrodes, in known ways to capture cardiac data signals.

Once the electrodes 204 are placed on a patient and coupled to the monitoring device 400, the device 400 can obtain cardiac information from the patient. The obtained cardiac information can correspond to propagated electrical signals, as previously described, chemical compound concentrations, blood pressure, or other measurements indicative of cardiac health. Optionally, the obtained cardiac information can be processed by the monitoring device 400 before transmission to correspond to the patient's resting heart rate, active heart rate, electrical mapping of the heart, ectopy measurement, ectopy type, predicted three dimensional information of the patient's heart chamber, evaluation of dynamic changes, heart recovery period, etc. The raw or processed cardiac information can be stored locally in the device 400 on a temporary or non-temporary memory device 408 before transmitting, transmitted live, i.e., as the information is collected, or a combination of both. Optionally, the monitoring device 400 can include a data conditioner that makes the cardiac information easier to process on the data-computing device. Examples of data conditioners can include, but are not limited to, signal amplifiers, signal filters, data encoders, and data converters. Similarly, the cardiac information can be subjected to a data quality enhancement routine to eliminate redundancy. Since the collected cardiac information will tend to be collected as analog signals, the data conditioner can be an analog to digital converter to allow for easier transmission and analysis of the cardiac information.

The collected cardiac information 300, shown as a waveform in FIG. 3, can include two or more data points for statistical review and analysis. As depicted in FIG. 3, eleven collected data points can include: two points of height and length of the P wave 302 (morphologic change); two points of length and height of the P-R interval 304; two points for QRS wave 306 width and height change; one point for the shape of the QRS wave 306; two points for the S-T interval 308 and height; and two points for the T wave 310 height and length. Further data that can be collected from the point data can include the length of Q-T; the QTc; the T-P interval; change in sloping of the QRS wave 306 in a predetermined period of the polarization of the heart ventricle; change in shape of the T wave 310; change in shape of the P wave 302; and evaluation of the last 50 milliseconds of the QRS wave 306 with change and shape and slope with the end of polarization of the ventricular cavity. It should be appreciated that many more data points can be collected and analyzed according to the present invention, and that the described data points and relationships are exemplary only.

The collected data points 300 can be assessed for diagnostic and predictive tests focusing on specific heart disease detection. Some of the following heart diseases can be diagnosed and predicted using information from the collected data points. Coronary Artery Disease (CAD) can be predicted or diagnosed based on the ST segment evaluation in real time over a significant period of time. Congestive Heart Failure (CHF), either systolic or diastolic, can be predicted or diagnosed based on change in intervals of S-T segments 308, QRS wave 306 width and height, alternating patterns in QRS waves 306, and alternating patterns in T waves 310 during daily activities of living (ADL's), as well as heart rate variability measurement. Cardiomyopathy can be predicted or diagnosed based on QRS wave 306 width and changes with ADL's as well as changes in T wave 310 morphology. Stroke can be predicted or diagnosed based on atrial arrhythmias, which are typically asymptomatic. Sudden cardiac death can be predicted or diagnosed based on ventricular ectopy with heterogeneity of the origins evaluated at 360°, evaluated origins of ectopy, runs vs. frequency of ectopy, changes in frequency of ectopy associated with activities and changes in repolarization (T waves). Atrial arrhythmias can be predicted or diagnosed based on change in P wave 302 intervals, change in P-R intervals 304, or findings of intraventricular or AV nodal blocks.

Once the cardiac information 300 is ready to be transmitted, the monitoring device 400 or a connected communication device 602 (shown in FIG. 6) can transmit the cardiac information 300 in a raw or processed form to the data-computing device 504. Transmission of the information 300 can be accomplished by any method that allows electronic data transmission such as cellular transmission, radio transmission, transmission over the Internet, transmission over a Wireless Fidelity (WiFi) network, etc. Such types of electronic data transmission can be directly to the data-computing device 504 or through an intermediary such as a mobile carrier, server, network, cloud, etc. The transmission of the information 300 can also be accomplished by storing the data on a memory unit or device, such as a hard drive or solid state memory drive, and physically delivering the memory module to the data-computing device 504 physically located either locally or at a remote data location. Once the cardiac information is transmitted to and received by the data-computing device, the cardiac information can be stored and analyzed.

The cardiac information 300 can be sent by itself, or can also be paired with personal information about the patient that was the source of the cardiac information 300. The personal information can be a unique device identifier that is associated with the monitoring device 400 as well as medically significant demographic attributes about the patient 202 associated with the monitoring device 400 such as age, sex, state of residence, height, weight, body temperature, ethnic background, nationality, etc. The demographic attributes can be chosen such that the patient's identity cannot be ascertained from the demographic attributes alone. To protect the patient's privacy, personal health information (PHI) can be stored in an encrypted form that would prevent the patient's identity from being easily ascertained or can be excluded from being transmitted to the data-computing device 504.

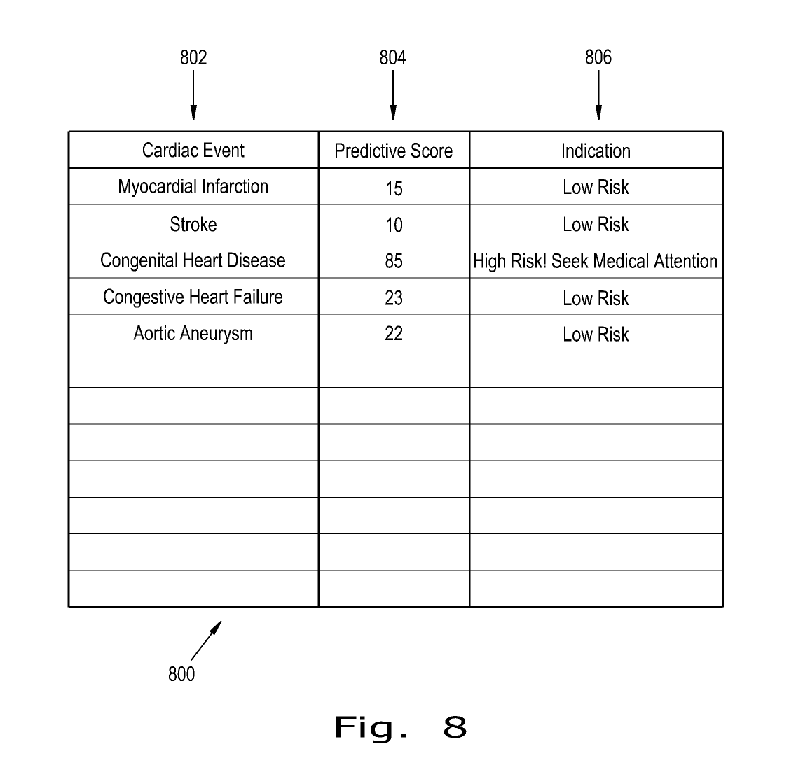

Once the cardiac information 300 and any personal information is transmitted to the data-computing device 504, the cardiac information 300 can be analyzed. For example, the cardiac information 300 transmitted to the data-computing device 504 can be electrocardiogram, commonly known as either ECG or EKG, signals that are transmitted as waveforms 300, such as those shown in FIG. 3. The ECG waveforms 300 can be analyzed at the data-computing device 504 to produce a three dimensional map of the electrical signals in the heart. The transmitted cardiac information 300 can be compared 114 with baseline data that has been stored 112 and established via clinical testing to build 108 an indication report for an individual patient indicating the risk that the patient experiences future cardiac disease. The baseline data can be used to build a predictive model for use in statistical analysis of cardiac information to determine whether a patient is truly at risk for various cardiac conditions, allowing for early intervention or treatment to be administered. The indication report, an example one being shown in FIG. 8, can be based on and/or include the predictive score to display the likelihood of various cardiac events occurring. The predictive model used to build 108 the indication report can be a compilation of baseline data that has been analyzed for patterns. For example, the predictive model can be an algorithm constructed from the baseline data and compared 114 to transmitted cardiac information to determine 118 a predictive score on a scale of 1 to 100, with a 1 signifying a low risk of a cardiac event occurring in the near future or in the process of occurring and a 100 signifying an extreme risk of a cardiac event occurring in the near future or in the process of occurring. The predictive model can be constantly updated as new cardiac information becomes available for analysis, allowing for a patient's predictive score to be constantly updated and monitored as new statistical analysis techniques are developed. It is also contemplated that the predictive model can be specific to a particular patient, e.g., a “normal” ECG waveform that is constructed from a specific patient's previously collected cardiac information and compared to incoming ECG waveforms to determine if there are deviations that might indicate a future cardiac event.

The baseline data can be stored 112 in a baseline data repository in a memory unit of the monitoring device 400 or at a remote data center 502 (shown in FIG. 5) and be built from previously conducted clinical trials and analyses. The baseline data can also be sourced from cardiac information that is transmitted from multiple patients. For example, transmitted cardiac information can be frequently added to the baseline data from a controlled study group of patients or the general population. The transmitted cardiac information can be stored 120 as a patient cardiac state, signifying the state of the patient's cardiovascular function at the time of recording, and be associated 122 with a cardiac event that the patient experiences. The cardiac event can be determined by the monitoring device 400 or treatment device 600 using known data analysis techniques. The monitoring device 400 or treatment device 600 can also be programmed to have a stored time interval that can be used to help determine whether a cardiac state was a significant indicator of a cardiac event. For example, the monitoring device 400 can determine a cardiac event has occurred and compare the timestamp of the cardiac event occurring to the timestamp of the previously determined patient cardiac state to determine 124 whether the cardiac event occurred within a stored time interval, such as 24 hours, after storing the patient cardiac state. Alternatively, the monitoring device 400 or treatment device 600 can recall all cardiac information that was collected in a stored time interval, such as 24 hours, prior to the cardiac event.

After determining 124 a cardiac event has occurred in the stored time interval following a stored patient cardiac state, the cardiac event determination and patient cardiac state can be added 126 to the baseline data to include more data that can be analyzed to determine reliable predictors of cardiac events. Common cardiac information characteristics that accompany general or specific cardiac events from multiple patients can then be identified in the baseline data for use in predictive modeling and scoring. In this sense, the baseline data can be constantly updated with more information to build a statistical model that can improve the accuracy of diagnoses based on the predictive model produced.

Using a remote data center 502 communicating with the monitoring device 400 allows utilization of large amounts of computing power without the size requirements of a wearable device. For example, high quality data transmitted to the remote data center 502 from the monitoring device 400 can be analyzed by one or more data-computing devices 504, such as servers or so-called “supercomputers,” to rapidly produce a three dimensional predicted image of the patient's heart without using invasive instrumentation. The produced three dimensional image can give insight into: the positioning of extra beats as compared to the normal path which electricity uses in the heart; frequency model building for multifocal versus unifocal on some numbered regions of activity; scar tissue locations in patients with previous heart attack (infarct); a predicted size of an infarct which can be used to build a predictability model for VT (life threatening arrhythmias); an atrial fibrillation or stroke predictability model based on the origin around pulmonary veins or build other segments (created by experience by data) by adaption; predicted mass of the heart; predicted size of the cavities; assessment of His to ventricle (H-V) interval; and assessment of heart rate variability and corresponding models with follow up questionnaires sent to patients to increase accuracy. It should be appreciated that while the data-computing devices 504 are shown as servers that include a central processing unit (CPU), the data-computing device according to the present invention can be any type of analog and/or electronic device that is capable of performing data computation functions.

Once an indication report has been built 108, whether based on a predictive score or not, the indication report 108 can be output 110 to the monitoring device 400, the treatment device 600, a health information storage medium such as a web environment, a device with a CPU, a memory unit, etc. The indication report, which can be based on the predictive score(s) determined, can correspond to specific cardiac diseases a patient might be susceptible to or currently experiencing. The indication report can be in any format that can be readily utilized and/or interpreted by a user such as a health professional, patient or insurance company, with or without the aid of an electrical processing circuit.

Optionally, an alerting signal can be outputted 128 by the data-computing device 504 to an alerting device 604. The alerting device 604 can be incorporated within the monitoring device 400 or treatment device 600, or can be a different device such as a physician's pager. The alerting device 604 can also be a device with a CPU such as a server, a personal computer, a laptop, a table computer, etc. that can include a stored program which displays an alert signified by the alerting signal or otherwise alerts a user that an alerting signal has been output 128. The alerting signal can be output when the predictive score determined by the data-computing device 504 is at or above a certain threshold, corresponding to a high risk of a cardiac disease or a cardiac event. The alerting signal can, for example, cause the alerting device 604 to issue a warning to the patient or the patient's doctor that medical attention should be sought. The warning can be any type of change that is perceptible to a person, such as a warning light, a light color change, an emitted sound, a vibration of the alerting device, etc. Optionally, the alerting device 604 can have an Internet connection to allow the alerting device 604 to send an electronic mail or other message warning to pre-designated recipients. The alerting device 604 can also be configured so that once the alerting signal is received, the alerting device 604 will continue to issue the warning at a preset interval until reset.

A treatment signal can also be output 130 from the data-computing device 504, in addition to or instead of the output alerting signal. For example, the monitoring device, which can be included as part of a pacemaker implanted within a patient, can output cardiac information to a data-computing device for analysis. Once analysis is complete and it is determined that the patient is experiencing a cardiac event, the data-computing device can send the treatment signal to the pacemaker to adjust the timing of corrective shocks delivered by the pacemaker to the patient's heart. A treatment signal can also be utilized in a defibrillator to automatically administer shocks to a fibrillating patient to stop the fibrillation. These examples of treatment signals are for illustration only, and any treatment signal can be output to control various medical devices to effectuate treatment of conditions that are diagnosed from cardiac information analyzed by the data-computing device.

In another embodiment of the present invention, a method for predicting or diagnosing heart disease can include the steps of storing baseline data at a data center, receiving cardiac information at the data center that is transmitted from a monitoring device, comparing the received cardiac information to the baseline data, producing a predictive score based on the comparison of the received cardiac information to the baseline data, and outputting an indication report based on the predictive score. If desired, the data center can add the received cardiac information and analysis to the stored baseline data. The data center can be a baseline data repository previously described or other hardware that is capable of storing the previously described baseline data. It should be appreciated that a “data repository,” as described herein, can refer to an electronic database or any other type of device that can be used to store data. The data center can also output an alerting signal to an alerting device, as previously described. Further, the data center can also output a treatment signal to a treatment device, as previously described.

In yet another embodiment of the present invention, a method for predicting or diagnosing cardiac disease can include the steps of providing a monitoring device that is configured to be attached to a patient, obtaining cardiac information using the monitoring device, providing baseline data to the monitoring device, comparing the obtained cardiac information to the baseline data, and outputting an indication report from the monitoring device. In this embodiment, it is contemplated that the monitoring device can have sufficient memory and processing power to store the baseline data and compare the baseline data to the obtained cardiac information in order to output an indication report. The indication report or cardiac information can be output to a remote data center. The remote data center can update the baseline data that is provided to the monitoring device at preset intervals, based on a request sent to the remote data center from the monitoring device or based on the indication report that is sent to the remote data center from the monitoring device.

Now, referring specifically to FIG. 4 there is illustrated a block diagram of monitoring device 400 in the form of electrical components that can be utilized to receive signals, digitize information in the signals, store the digitized information and make that information available for processing by other devices not illustrated, but known in the art. Monitoring device 400, includes a power source 402, a switch 404, a controller 406, a memory device 408, at least one parent signal receiver 410, and at least one child signal receiver 412. A power conductor system 414 provides electrical energy to each of the components. Data buses 416 and 418 allow transfer of digital information to and from the components respective coupled thereto.

Signal receivers 410 and 412 have signal lines 420 and 422 respectively that are coupled to sensors that are not illustrated, but are known to those skilled in the art. Signal lines 420 and 422 are each multiple signal lines, with each line being coupled to a sensor that is assigned to a location on a patient. The electrical signal may be passed through a filtering network to enhance the signal and/or diminish noise in the signal. It is also contemplated that digital filtering techniques may be used relative to the digitized data gathered from the electrical signals that originate with the sensors.

Digitized information gathered by signal receivers 410 and 412 are passed to controller 406 by way of data bus 418. Controller 406 may assign additional information to the digital information such as a time stamp and then stores the information in memory device 408. Controller 406 may be connected to a computing device, not illustrated, for the purpose of passing on the data stored in memory device 408. The data connection may be by way of a wired interface, a wireless interface or any data transferring technique. It is also possible that memory device 408 may be a removable memory device allowing the data to be moved by a physical disconnection from monitoring device 400 and the coupling of it to the computing device.

Referring now to FIG. 5, a patient is shown wearing the treatment device 600 which includes monitoring device 400. As can be seen, the monitoring device 400 is connected to the communication device 602 and electrodes 204 placed on the patient. After the monitoring device 400 has collected cardiac information via the electrodes 204, the monitoring device 400 can communicate with the remote data center 502 utilizing the communication device 602, with such communication being illustrated in FIG. 5 as cell signal waves. The cell signal waves can be received at the remote data center 502 by a data receiver 506 and transferred to one or more of the data-computing devices 504 for analysis using the baseline data and established predictive model and/or algorithm to build an indication report. Once the indication report is built, the indication report can be sent back to the communication device 602 by a data transmitter 508 connected to the data-computing device(s) 504 using cell signal waves. The monitoring device 400 and/or treatment device 600 can then display the indication report and alert the patient of an increased risk of a pending cardiac event. Further, the treatment device 600 can be activated with a treatment signal if it is determined that the patient is experiencing a cardiac event, such as atrial fibrillation. The treatment signal can be received by the treatment device 600 and be instructions for the treatment device 600 to follow a pre-programmed treatment protocol and/or a treatment protocol that is included in the treatment signal. While the communication device 602 is shown communicating with the data receiver 506 and data transmitter 508 using cell signal waves, it is also contemplated that the communication device 604 can communicate with the data receiver 506 and data transmitter 508 via WiFi signals, the Internet, or any other type of electronic communication. It can therefore be seen how the monitoring device 400 and treatment device 600 can utilize the communication with the remote data center 502 to not only add to the baseline data, but also treat the patient in the event that a cardiac event is detected when analyzing the transmitted cardiac information.

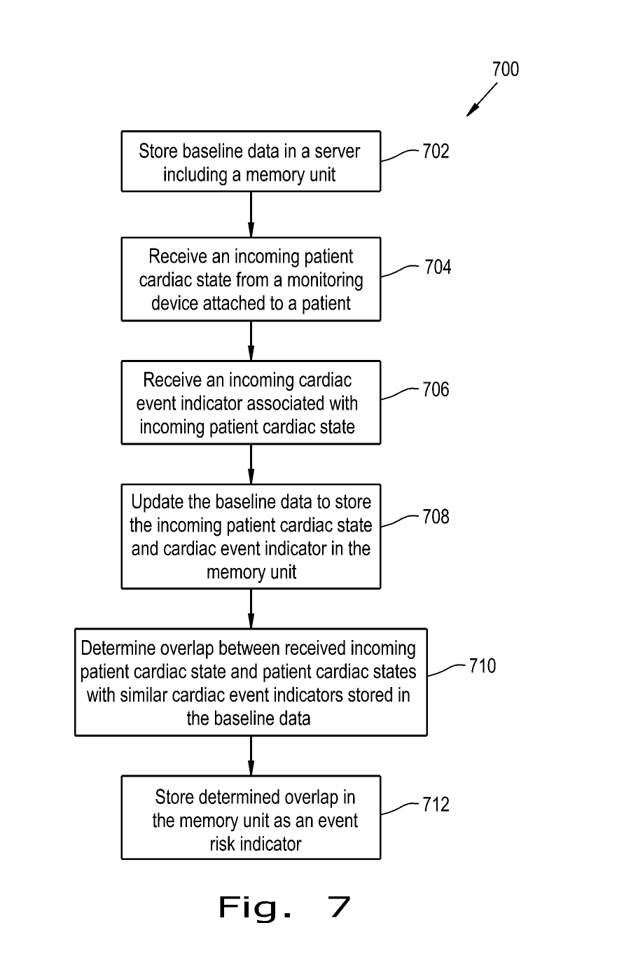

Referring now to FIG. 7, another embodiment of a method 700 according to the present invention is shown in flow chart form. The method 700 can be for maintaining a cardiac disease data repository, such as one stored at the remote data center 502 illustrated in FIG. 5. Baseline data is stored 702 in a memory unit 510 of a server, such as server 504 shown in FIG. 5. The baseline data includes multiple patient cardiac states, as previously described, and a cardiac event indicator that is associated with at least one of the patient cardiac states. For example, a stored patient cardiac state can be an ECG waveform of a patient that experienced a myocardial infarction within 6 hours of the ECG waveform being recorded, with the associated cardiac event indicator being a tag or other type of information signifying that a myocardial infarction occurred within a defined time period of the ECG waveform being recorded. The remote data center 502 then receives 704 an incoming patient cardiac state from a monitoring device attached to a patient, such as monitoring device 400 shown in FIGS. 4-5. A server, such as data-computing device 504, at the remote data center 502 receives 704 the incoming patient cardiac state. The server 504 also receives 706 an incoming cardiac event indicator that is associated with the incoming patient cardiac state, as previously described. For example, the server 504 can receive an incoming patient cardiac state that was recorded as a patient was experiencing a myocardial infarction, with the incoming cardiac event indicator associated with the incoming patient cardiac state signifying that the incoming patient cardiac state was recorded as the patient was experiencing a myocardial infarction. In other words, the incoming cardiac event indicator can be a part of the incoming patient cardiac state as a tag or other signifying information. The incoming cardiac event indicator can also be received by the server 504 after the incoming patient cardiac state was received, such as when the monitoring device 400 measures a cardiac event occurring within a predetermined monitoring interval, such as one hour, of the incoming patient cardiac state. To assist in associating incoming cardiac event indicators with their associated incoming patient cardiac state, each incoming patient cardiac state can be assigned a unique identifier number, with the associated incoming cardiac event indicator also sharing this unique identifier number or a similar one, with such techniques for pairing two different data events being known in the art. Once the incoming patient cardiac state is received 704 and the associated incoming cardiac event indicator is also received 706 by the server 504, the baseline data stored in the memory unit 510 is updated 708 to store the incoming patient cardiac state and the associated cardiac event indicator, incorporating the incoming patient cardiac state and the associated cardiac event indicator in the baseline data to increase the amount of data points in the baseline data for analysis.

Once the baseline data is updated 708, the received incoming patient cardiac state can be compared with previously stored patient cardiac states having similar associated cardiac event indicators to determine 710 overlap between patient cardiac states having similar associated cardiac event indicators. For example, if the received incoming patient cardiac state has an associated cardiac event indicator signifying a myocardial infarction, the received incoming patient cardiac state can be compared with other patient cardiac states associated with myocardial infarction to determine 710 overlap between patient cardiac states associated with myocardial infarction. The determined overlap between the patient cardiac states associated with, for example, myocardial infarction can then be stored 712 in the memory unit 510 as an event risk indicator of myocardial infarction. The stored event risk indicators can establish common cardiac states associated with a specific cardiac event and allow for the utilized predictive model to be updated with an increasing amount of data points and outcomes to increase the quality of the predictive model.

Referring now to FIG. 8, an example indication report 800 that can be output according to an embodiment of a method according to the present invention is shown. The indication report 800 can be displayed, for example, by the monitoring device 400 and/or treatment device 600. As can be seen, the indication report 800 can be a table with a cardiac event column 802, a predictive score column 804, and an indication column 806. The cardiac event column 802 can list various cardiac events that are of analytical interest, shown as being myocardial infarction, stroke, congenital heart disease, congestive heart failure, and aortic aneurysm. The displayed and analyzed cardiac events can be tailored to a specific patient based on the patient's health profile and family history, and it should thus be appreciated that the shown cardiac events are exemplary only. Each specific cardiac event can be given a corresponding predictive score, displayed in the predictive score column 804, that is determined according to a method of the present invention by analyzing cardiac information from the patient and performing statistical analysis of the cardiac information to determine if the patient is at risk of a cardiac event occurring. As can be seen, the predictive scores shown in the predictive score column 84 range from 10 to 85, with the predictive scores of 10, 15, 22 and 23 being relatively low and indicating a low risk of the respective cardiac event occurring, as shown by the accompanying indication for each cardiac event in the indication column 806. The predictive score of 85 for congenital heart disease, however, is determined to be 85 from analysis of the cardiac information from the patient, corresponding to a high risk of the patient having a congenital heart defect. This is displayed in the indication column 806 as a high risk, and advises the patient to seek medical attention. The indication report 800 can therefore be a useful tool for patients and healthcare professionals to determine what cardiac events the patient is at a significant risk of developing or experiencing, and do further diagnostics to determine if treatment is necessary.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of predicting cardiac disease, comprising the steps of:
attaching a monitoring device to a patient;
obtaining cardiac information from the patient using said monitoring device;
transmitting said obtained cardiac information from said monitoring device to a data-computing device;
analyzing said transmitted cardiac information using said data-computing device and a predictive model;
building an indication report from said analyzed cardiac information; and
outputting said indication report.

2. The method according to claim 1, wherein said analyzing step comprises:
storing baseline data in at least one of a local and remote data center utilized by said data-computing device;
comparing said transmitted cardiac information to said baseline data; and
determining a predictive score based on said comparison.

3. The method according to claim 2, further comprising the step of outputting an alerting signal from said data center to an alerting device based on said predictive score.

4. The method according to claim 2, further comprising the step of outputting a treatment signal from said data center to a treatment device based on said predictive score.

5. The method according to claim 4, wherein said treatment device is at least one of a pacemaker, a defibrillator, and a medication pump.

6. The method according to claim 4, wherein said treatment device is connected to said monitoring device.

7. The method according to claim 6, wherein said cardiac event is at least one of a myocardial infarction, a stroke, an atrial fibrillation, an aortic aneurism, and an aortic dissection.

8. The method according to claim 2, further comprising the steps of:
storing said transmitted cardiac information as a patient cardiac state;
determining if a cardiac event has occurred in a stored time interval following storing said patient cardiac state; and
adding said cardiac event determination and said patient cardiac state to said baseline data.

9. The method according to claim 8, further comprising the step of associating said patient cardiac state with an occurred cardiac event prior to adding said cardiac event determination and said patient cardiac state to said baseline data.

10. The method according to claim 1, wherein said indication report is output to at least one of said monitoring device, a web environment, a memory unit, and said data-computing device.

11. The method according to claim 1, wherein said obtained cardiac information includes at least one of a length of a P wave, a height of a P wave, a length of a P-R interval, a height of a P-R interval, a QRS wave width, a QRS wave height, a QRS wave shape, an S-T interval, an S-T height, a T wave height, a T wave length, a Q-T length, a QTc, a T-P interval, a slope change of a QRS wave in a predetermined period of a heart ventricle polarization, a T wave shape change, and a P wave shape change.

12. The method according to claim 1, wherein said transmitting, analyzing, and outputting steps occur at predetermined time intervals.

13. The method according to claim 1, wherein said analyzing step includes using an algorithm to determine cardiac event indicators.

14. The method according to claim 13, wherein said algorithm is modified by subsequently transmitted cardiac information.

15. A method of maintaining a cardiac disease data repository, comprising the steps of:
storing baseline data in a server including at least one memory unit, said baseline data including a plurality of patient cardiac states and a cardiac event indicator associated with at least one of said patient cardiac states;
receiving an incoming patient cardiac state from a monitoring device attached to a patient, said server receiving said incoming patient cardiac state;
receiving an incoming cardiac event indicator associated with said incoming patient cardiac state, said server receiving said incoming cardiac event indicator; and
updating said baseline data to store said incoming patient cardiac state and said associated cardiac event indicator in said at least one memory unit.

16. The method according to claim 15, wherein said incoming cardiac event indicator associated with said incoming patient cardiac state is measured by said monitoring device simultaneously with said incoming patient cardiac state.

17. The method according to claim 15, further comprising the step of determining overlap between said received incoming patient cardiac state and patient cardiac states with similar cardiac event indicators stored in said baseline data.

18. The method according to claim 17, wherein said determined overlap is stored in said at least one memory unit as an event risk indicator.

19. The method according to claim 15, wherein said incoming cardiac event indicator associated with said incoming patient cardiac state is measured by said monitoring device within a predetermined monitoring interval of said incoming patient cardiac state.
